(19) United States
(12) Patent Application Publication
(10) Pub. No.: US 2007/0048377 A1
(43) Pub. Date: Mar. 1, 2007

(54) Drug compositions containing controlled release hypromellose matrices

(76) Inventors: Ali Rajabi-Siahboomi, Lansdale, PA (US); Kurt Alan Fegely, Greenville, PA (US); Cara Young, Blue Bell, PA (US); Pankaj Rege, Glen, PA (US)

Correspondence Address: LUCAS & MERCANTI, LLP, 475 PARK AVENUE SOUTH, 15TH FLOOR, NEW YORK, NY, 10016, US

(21) Appl. No.: 11/510,359

(22) Filed: Aug. 25, 2006

Related U.S. Application Data
(60) Provisional application No. 60/711,724, filed on Aug. 26, 2005.

(51) Int. Cl. A61K 9/22

(52) U.S. Cl. 424/468

(57) ABSTRACT

This invention is directed to a controlled release formulation for an oral dosage form that is formulated into a swellable, hydrophilic matrix. The controlled release formulation contains a mixture of hypromellose and polyvinyl acetate phthalate and allows pharmaceutically active ingredients combined therewith to be released in a controlled release manner.

### CROSS REFERENCE TO RELATED APPLICATIONS

This application claims benefit of priority under 35 U.S.C. 119(e) of U.S. Provisional Application Ser. No. 60/711,724 filed on Aug. 26, 2005, the contents of which are incorporated herein by reference.

### FIELD OF THE INVENTION

This invention is directed to controlled release pharmaceutical formulations. In particular, the invention is directed to hypromellose-containing powder mixtures which can be used to make controlled release oral solid dosage forms containing a hydrophilic, swellable matrix.

### BACKGROUND OF THE INVENTION

The advantages of controlled release oral solid dosage forms are well known in the pharmaceutical arts. Some of the advantages include once daily dosing, the ability to maintain a desirable blood level of an active pharmaceutical ingredient (hereinafter “API”) over an extended period, such as twenty four hours, minimizing the peak to trough variations in plasma concentrations, etc. Studies also show that patient compliance is increased by reducing the number of daily dosages. While many controlled and sustained release formulations are already known, there continues to be a need to provide improvements and alternatives.

Some efforts in the field of controlled release include those which have incorporated the use of hydrophilic swellable matrices. Drug release from the matrix is accomplished by swelling, dissolution, diffusion and/or erosion. The major component of these systems is a hydrophilic polymer. In general, diffusivity is high in polymers containing flexible chains and low in crystalline polymers. With changes in morphological characteristics, the mobility of the polymer segments will change and diffusivity can be controlled. Often, the addition of other components, such as a drug, another polymer, soluble or insoluble fillers, or solvent, can alter one or more properties of the final product such as the intermolecular forces, free volume, glass transition temperature. Each variable can have an effect on the release rate of the drug from the matrix.

For example, U.S. Pat. No. 6,090,411 describes monolithic tablets containing a swellable hydrodynamically balanced monolithic matrix tablet. The swellable hydrophilic matrix tablet is said to deliver drugs in a controlled manner over a long period of time and be easy to manufacture. The drug is disposed in the HPMC or polyethylene oxide-based matrix, in the presence of a salt.

In another example of such matrix-based tablets, U.S. Pat. No. 6,875,793 discloses controlled release tablets containing a sulfonylurea. The rate controlling feature is based on a matrix containing a polysaccharide blend of materials such as locust bean gum or xanthan gum. The API is dissolved in a suitable solvent before being blended with rate controlling matrix.

In spite of the foregoing, there is also a need in the industry to provide further improvements in the field of controlled release solid dosage forms. For example, it has determined that it would be beneficial to provide the artisan with a pre-mix or partially pre-mixed oral solid dosage formulation which the artisan can quickly adopt for use in the production of new compressed tablets. The present invention addresses this need.

### SUMMARY OF THE INVENTION

In one aspect of the invention, there is provided a controlled release formulation for use in oral dosage forms. The controlled release formulation includes a mixture of hypromellose and an anionic polymer such as polyvinyl acetate phthalate (hereinafter PVAP). The PVAP is present in the mixture in an amount which is effective to provide controlled release of a pharmaceutically active ingredient when the mixture is compressed into a swellable, hydrophilic matrix. In further aspects, an auxiliary anionic polymer is included in combination with the PVAP and hypromellose. The controlled release of the active pharmaceutical ingredient (API) afforded by the inventive mixture is observed in dissolution media simulated to represent the pH of physiological fluids present over the entire gastrointestinal tract.

The inventive mixture is preferably in powder form and can preferably include an API and/or nutritional supplement. For purposes of the present invention, API shall be understood to include not only pharmaceutical ingredients but also nutritional supplements and/or any other agent or biologically active ingredient suitable for delivery by oral solid dosage forms.

In other aspects of the invention, there are provided oral solid dosage forms containing an API, the inventive powder mixture, preferably in the form of a swellable hydrophilic matrix, and methods of preparing the same.

As a result of the present invention, there are provided new controlled release formulations for the modulation of drug release from HPMC (hypromellose) matrices. It has been surprisingly found the artisan can include PVAP into the matrix to control the release of the API over not only dissolution media intended to simulate the alkaline environments of the GI tract but also dissolution media intended to simulate the neutral and acidic regions of the GI tract as well. In the past, PVAP was believed to be primarily useful for as an enteric coating for compressed tablets. According to the Handbook of Pharmaceutical Excipients Fourth Ed., 2003, PVAP dissolves along the entire length of the duodenum. It was therefore quite surprising that it could be combined with HPMC or hypromellose to modulate the release of API's in neutral and acid environments as well. The combination provides a robust matrix for a full range of highly soluble to practically insoluble active pharmaceutical ingredients.

### BRIEF DESCRIPTION OF THE DRAWINGS

FIG. 1 is a gel formation graph corresponding to Example 2.

FIG. 2 is a graph which plots a tablet resistance/force of penetration vs. time, corresponding to Example 3.

FIG. 3 is a graph showing the mass loss of the formulations described in Example 4.

FIG. 4 is a graph showing the liquid uptake profile of the formulations described in Example 4.

FIG. 5 is a graph showing the dissolution of various Verapamil HCL containing solid dosage forms prepared in accordance with the present invention and Example 6.

### DETAILED DESCRIPTION OF THE INVENTION

In a first aspect of the invention, there is provided a controlled release formulation for use in oral dosage forms. The formulation includes a mixture containing hypromellose and polyvinyl acetate phthalate. The amount of PVAP included in the inventive mixture is an amount which is effective to provide controlled release of a pharmaceutically active ingredient in vitro when the mixture is compressed into a swellable, hydrophilic matrix.

Matrix systems are well known in the art. In a typical matrix system, the drug is homogenously dispersed in a polymer in association with conventional excipients. This admixture is typically compressed under pressure to produce a tablet. The API is released from the tablet by diffusion and erosion. Matrix systems are described in detail by (i) Handbook of Pharmaceutical Controlled Release Technology, Ed. D. L. Wise, Marcel Dekker, Inc. New York, N.Y. (2000), and (ii) Treatise on Controlled Drug Delivery, Fundamentals, Optimization, Applications, Ed. A. Kydonieus, Marcel Dekker, Inc. New York, N.Y. (1992), the contents of both of which are hereby incorporated by reference.

When the tablet is exposed to aqueous media, such as in the gastrointestinal tract, the tablet surface wets and the polymer begins to partially hydrate forming an outer gel layer. This outer gel layer becomes fully hydrated and begins to erode into the aqueous fluids. Water continues to permeate toward the core of the tablet permitting another gel layer to form beneath the dissolving outer gel layer. These successive concentric gel layers sustain uniform release of the API by diffusion from the gel layer and exposure through tablet erosion. In the case of the mixtures of the present invention, when included in a compressed tablet matrix, the hypromellose provides a hydrophilic swellable structure capable of functioning as the gel layer while the PVAP portion of the matrix provides means to modulate the thickness of gel formation, hydration rate and water uptake of the tablets. In this way, the drug release is controlled.

For purposes of the present invention, “controlled release” shall be understood to relate to the release of an API from a matrix prepared from the inventive mixture. “Controlled” refers to the ability of the artisan to provide a dosage form with the API being released therefrom in vitro and/or in vivo at a predictable and substantially repeatable rate. As will be appreciated by those of ordinary skill, API release patterns which are “controlled” are not limited to extended or prolonged release profiles. Thus, by “controlled” release of the API, it is to be understood that the API is released predictably after ingestion and/or a period of time which may be extended or otherwise in a manner which is advantageous for the patient receiving the API within acceptable statistical measurements of deviation for the art.

In the case of the present invention, the controlled release of the API can be observed in vitro in dissolution media which simulate the pH of physiological fluids found along the gastrointestinal tract. Formulations of the present invention are associated with API release profiles which can begin within minutes of ingestion, up to and including 24 hours or longer.

The type of hypromellose included in the formulations of the present invention include all such types recognized in the art as being pharmaceutically acceptable. Hypromellose is also known in the art as hydroxypropylmethylcellulose or HPMC and is available from several chemical companies under different trade names. For example, HPMC is available from the Dow Chemical Company under the trade name Methocel®. HPMC's are classified based on their type and level of substitution as well as their solution viscosity at 2% w/v in water at 20° C. A non-limiting list of suitable grades of HPMC includes Methocel K100LV, E-50, K4M, K15M, K100M E4M, E10M, or any grade with a viscosity between 50 and 100,000 centipoise at 20° C.

The amount of hypromellose included in the powder mixtures of the present invention can broadly range from about 8 to about 60% by wt. Preferably, the amount of hypromellose included is from about 15 to about 45% by wt., while in more preferred aspects of the invention, the amount of hypromellose is from about 25 to about 35% by wt. of the powder mixture. In most aspects of the invention, the hypromellose is combined with the PVAP or other anionic polymer, optionally included API, and other carrier materials, and then either direct compressed or wet granulated, fluid bed dried, blended and compressed into a tablet dosage form.

The preferred anionic polymer included in the formulations of the present invention is polyvinyl acetate phthalate which is available, for example, from Colorcon of West Point, Pa. The PVAP included in the present invention may also be co-processed with titanium dioxide, available from Colorcon as PVAP-T. The amount of PVAP and, if desired, auxiliary anionic polymer(s) included in the mixtures of the present invention is described as an amount which is effective to provide controlled release of a pharmaceutically active ingredient when the mixture is compressed into a swellable, hydrophilic matrix. While this amount will vary somewhat according to the needs of the artisan, presence or absence of other ingredients, etc., the amount included will generally be from about 4 to about 60% by wt. of the mixture, preferably from about 8 to about 45% by wt. of the mixture, and more preferably from about 15 to about 35% by wt. of the mixture. As mentioned above, one of the keys to the controlled release aspects of the invention is the use of PVAP to control the release of the API in the GI tract, especially in the acid and neutral regions thereof. In most aspects of the invention, the PVAP (an anionic polymer), will constitute the majority of the anionic polymers included.

In further aspects of the invention, the auxiliary anionic polymer is selected from among pharmaceutically acceptable anionic polymers such as and without limitation, sodium carboxymethylcellulose, sodium alginate, xanthan gum, Carbopol (cross-linked acrylic acid polymers), cellulose acetate phthalate, hydroxypropylmethylcellulose phthalate, methacrylic acid copolymer, hydroxyppropylmethyl acetate succinate, and mixtures thereof.

In one aspect of the invention, the hypromellose and PVAP are preferably combined in the form of a mixture, prior to being combined with the API. The mixture can be obtained by dry blending the two ingredients, i.e. hypromellose and PVAP, until an intimate mixture or a substantially homogeneous combination of the ingredients is obtained. It will be understood that those other art-recognized methods of blending can also be employed. The auxiliary anionic polymer can be combined with the PVAP either separately prior to blending with the hypromellose or as part of a tertiary mixture. For ease of discussion, the mixture of the hypromellose and PVAP and, if included, auxiliary anionic polymer, shall be referred to as the “preblend”.

In an alternative aspect, the preblend is made with the API first being combined with the HPMC or the PVAP and optional filler or diluents before being combined with the other mixture components.

It is contemplated that in many preferred embodiments that the powder-based mixtures of the present invention will preferably include a pharmaceutically active ingredient or a nutritional supplement. There are no known limitations on the type of the API which can be included in the powder mixtures and/or hydrophilic matrixes including the same other than that the API must be suitable for inclusion in a hydrophilic matrix and that it must be capable of being included in a solid oral dosage form.

The preblend can be combined with the API in any art-recognized fashion. In some preferred aspects of the invention, the preblend is combined with the API using wet granulation techniques. Other aspects of the invention call for dry blending all components of the oral solid dosage form and using direct compression.

The following non-limiting list of API's is meant to be illustrative rather than restrictive of the API's suitable for inclusion in the powder mixtures of the present invention and/or oral solid dosage forms containing the same:
a) Analgesics such as codeine, dihydrocodeine, hydrocodone, hydromorphone, morphine, diamorphine, fentanyl, buprenorphine, tramadol, oxycodone, acetaminophen, aspirin, phenylbutazone, diflunisal, flurbiprofen, ibuprofen, diclofenac, indomethacin, naproxen, methadone, meloxicam, piroxicam, or azapropazone;
b) Antihistamines such as loratidine, diphenhydramine, etc.;
c) Antihypertensives such as clonidine, terazosin, acebutalol, atenolol, propranolol, nadolol, nifedipine, nicardipine, verapamil, diltiazem, lisinopril, captopril, ramipril, fosinopril, enalapril, etc.;
d) Antibiotics such as democlocycline, doxycycline, minocycline, tetracycline, ciproflaxacin, amoxicillin, penicillin, erythromycin, metronidazole, cephalosporins, etc.;
e) Bronchial/anti-asthmatic agents such as terbutaline, salbutamol, theophylline, etc.;
f) Cardiovascular products such as procainamide, tocainide, propafenone, etc.;
g) Central nervous system agents/ anti-anxiety agents/ antidepressants such as levodopa, fluoxitene, doxepin, imipramine, trazodone, fluphenazine, perphenazine, promethazine, haloperidol, oxazepam, lorazepam, diazepam, clonazepam, buspirone, etc.;
h) Anti-cancer agents such as melfalan, cyclophosphamide, fluorouracil, methotrexate, etc.;
i) Anti-migraine products such as sumatriptan, lisuride, etc.;
j) Gastrointestinal agents such as cimetidine, ranitidine, omeprazole, misoprostol, etc.; and
k) Oral anti-diabetic agents such as glipizide, gliboruride, etc.

The artisan will also appreciate that all pharmaceutically active salts or esters of the above and combinations of two or more of the above or salts or esters thereof are also contemplated as are those pharmaceutical agents currently known but not specifically mentioned. In most embodiments of the invention where the API is included, the pharmaceutically active ingredient makes up from about 0.001 to about 60% by weight of the mixture. Preferably, the API makes up from about 5.0 to about 40% by weight of the mixture, while amounts of from about 10 to about 30% by weight of the mixture are more preferred.

In a further aspect, the inventive mixtures and hydrophilic matrixes made therewith include an auxiliary hydrophilic cellulosic polymer. A non-limiting list of suitable auxiliary hydrophobic polymers includes hydroxypropylcellulose, hydroxyethylcellulose, polyvinyl acetate and mixtures thereof. Such auxiliary polymers can be present in amounts ranging from >0 up to about 100% by weight of the hypromellose content.

In a still further aspect of the invention, the hypromellose/PVAP powder mixtures can include one or more pharmaceutically acceptable excipients including but not limited to lubricants, flow aids, diluents, binding agents, disintegrants, binders, solubility enhancers, pH modulating agents, glidants, anti-adherents, etc. and mixtures thereof. Such materials can be present in amounts which range from about 0.001 to about 50% by weight of the total tablet weight. It will be understood that the sum of the individual excipients mentioned below will fall within the range provided.

Suitable lubricants include, for example materials such as stearic acid, metallic stearates (e.g. calcium, magnesium, sodium), polyxamer, polyethylene glycols, e.g. Carbowaxes, hydrogenated vegetable oils such as Sterotex, and mixtures thereof. Suitable flow aids include, for example colloidal silicon dioxide, talc, sodium stearyl fumarate (Pruv), sodium lauryl sulfate, etc. and mixtures thereof. The lubricant can be present in amounts ranging from about 0.1% to about 10%, preferably from about 0.2% to about 8%, and more preferably from about 0.25% to about 5%, of the total weight of the inventive compositions.

Suitable diluents include, for example, microcrystalline cellulose, lactose, dextrose, sucrose, dicalcium phosphate, pregelatanized starch, native starch, mannitol, talc and mixtures thereof. Other suitable inert pharmaceutical diluents include pharmaceutically acceptable saccharides, including monosaccharides, disaccharides or polyhydric alcohols.

If the inventive compositions are to be manufactured without a wet granulation step, and the final mixture is to be tableted, it is preferred that all or part of the inert diluent comprise an art recognized direct compression diluent. Such directed compression diluents are widely used in the pharmaceutical arts, and may be obtained from a variety of commercial sources. Examples include Emcocel. (microcrystalline cellulose, N.F.), Emdex. (dextrates, N.F.), and Tab-Fine (a number of direct-compression sugars including sucrose, fructose and dextrose), or others known to those of ordinary skill. The diluent can be present in amounts ranging from about 0.1% to about 60%, and preferably from about 5% to about 25% by weight of the total tablet weight.

Suitable disintegration aids include, for example, crospovidone, croscarmellose sodium, sodium starch glycolate, hydroxypropylcellulose (low-substituted), starch, calcium carbonate, carboxymethylcellulose calcium, and mixtures thereof. Disintegrants can be added at any suitable step during the preparation of a pharmaceutical composition made according to the methods of the present invention, but are preferably added prior to granulation or during the lubrication step prior to compression. In many aspects of the invention, the disintegrants are present in the range of about 0.5% to about 30%, preferably about 1% to about 10%, and more preferably about 2% to about 6%, of the total weight of the inventive compositions.

Suitable solubility enhancers include, for example, lecithin, poloxamer, polyoxyethylene fatty acid esters, sorbitan esters, and mixtures thereof. Suitable pH modulating agents include for example, citric acid, fumaric acid, tartaric acid, sodium citrate, sodium tartrate, sodium bicarbonate and mixtures thereof.

Suitable binding agents include those well known to those of ordinary skill which preferably impart sufficient cohesion to the powders to permit normal processing such as sizing, lubrication, compression and packaging, but still permit the tablet to disintegrate and the composition to dissolve upon ingestion, for example, povidone, acacia, gelatin, and tragacanth.

Other carrier materials (such as colorants, flavors and sweeteners) can be used in the preparation of the inventive pharmaceutical compositions of the present invention. Tablets made with the inventive compositions can be coated or uncoated. If film coated, materials such as Opadry® (Colorcon) or other art recognized film coating materials are useful.

The formulations according to the invention may be prepared by one or more of the following processes, although other, analogous methods may also be used. In one preferred aspect of the invention, however, the hypromellose and polyvinyl acetate phthalate are wet granulated with a pharmaceutically active ingredient. In other aspects, the primary ingredients, e.g. hypromellose and PVAP are dry blended optionally with the API and auxiliary excipients.

For purposes of illustration, a review of a suitable wet granulation is described below:

In wet granulation techniques, the desired amounts of API, PVAP and diluent are mixed together and thereafter combined with a solution containing a portion of the required hypromellose in the form of a solution under wet granulating conditions. The moistened mass is then dried, granulated and screened before being blended with the remainder of the hypromellose and other optional excipients such as magnesium stearate. The final blend is then ready for tableting.

In a still further embodiment of the invention, there are provided oral solid dosage forms containing the controlled release formulations described herein. Once the inventive powder mixtures are made, such as by dry blending or wet granulation, the mixtures can be compressed into tablets using art recognized techniques. Generally, the artisan can prepare an oral solid dosage form by providing a controlled release formulation described herein and compressing the formulation into an oral solid dosage form using a suitable tablet press.

### EXAMPLES

The following examples serve to provide further appreciation of the invention but are not meant in any way to restrict the effective scope of the invention.

### Example 1

To determine that the influence on the drug release is not due to the chemical interaction between Verapamil HCL and PVAP, following investigation was made.

### Determination of Verapamil Hydrochloride and Polyvinylacetate phthalate (PVAP) Chemical Interaction

a. Purpose—To determine if change in drug release is due to polymer drug interaction, where increasing PVAP would potentially cause decreased drug release due to binding with the drug.
b. Method—
   i. Dissolved 20 grams of Verapamil Hydrochloride in 52 grams of methanol to form a saturated solution.
   ii. Dissolved 10 grams of PVAP in 52 grams of methanol to form a saturated solution.
   iii. A clear solution was obtained for each sample.
   iv. 50 grams of each solution was combined and examined for the presence of a precipitate.
   v. Solution remained clear with no precipitate formed.
c. Conclusion
   A lack of chemical interaction has been shown between PVAP and the drug which is contra to some of previously published studies on the interactions of Verapamil HCl with enteric polymers. It also rules out that the reduction of drug release by using PVAP is due to a chemical interaction with Verapamil HCl.

### Example 2

### Investigation of Hydration Gel Formation of HPMC/PVAP Compacts

a. Composition
   PMC/PVAP compacts (5 g) were prepared using the Carver Press at the compaction force of 2500 pounds and the hold time of 15 s.

Compacts Compositions:

| | HPMC K100LV | PVAP 2138 | Lactose |
|---|---|---|---|
| A | 39.2 | | 60.8 |
| B | 39.2 | 60.8 | |
| C | 39.2 | 15.2 | 45.6 |
| D | 39.2 | 45.6 | 15.2 |

b. Method
   In order to evaluate the hydration/gel formation of each compact, they were placed in a beaker containing deionized water. All compacts floated on the surface. The tablets were removed from the beaker at predetermined time points (4, 8, 24 hours) and lightly patted with a tissue paper to remove excess water and were further subjected to textural analysis. The instrument was programmed so that the probe advanced towards the swollen tablet (centered under the probe) at a speed of 0.5 mm/s until the maximum force of 45N was achieved. The force-distance profiles associated with the penetration of the probe into the matrices were generated at a data acquisition rate of 200 points per second. Total swollen thickness was determined by measuring the total probe displacement recorded by the software.

Total Tablet Thickness (mm)

Tablet thickness (mm)

| Time (hr) | A | B | C | D |
|---|---|---|---|---|
| 0 | 8.591 | 10.4315 | 8.4515 | 9.5045 |
| 4 | 9.99 | 11.257 | 9.698 | 10.396 |
| 8 | 10.121 | 12.199 | 10.802 | 11.013 |
| 24 | 6.549 | 12.828 | 7.96 | 10.755 |

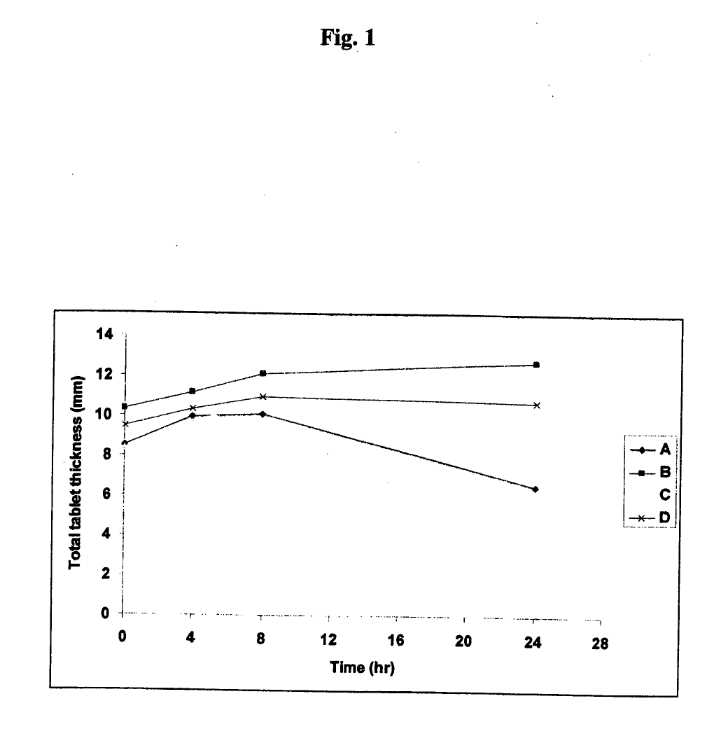

A plot of the above data is shown as FIG. 1.

c. Conclusion:
   Results indicate that increasing levels of PVAP (samples B and D) are more resistant to dissolution and dimensional change of the overall dosage form (gel layer and core) as evidenced by the similar values obtained for tablet thickness at the 8 and 24 hour time points. Contrastingly, tablets which contain higher levels of lactose when compared to PVAP provide reduced tablet thickness at the 8 and 24 time point's indicating a significant decrease in axial dimension due to dissolution/erosion of the gel layer and lactose from the hydrated core.

### Example 3

### Tablet resistance/Force of penetration Investigation

a. Composition
   PMC/PVAP compacts (5 g) with the compositions as in Example 2 were prepared using the Carver Press at the compaction force of 2500 pounds and the hold time of 15 s.
b. Method
   Same as the process of Example 2, the tablets were removed from the beaker at predetermined time points (4, 8, 24 hours) and lightly patted with a tissue paper to remove excess water and were further subjected to textural analysis. The instrument was programmed in such a way that the probe advanced towards the swollen tablet (centered under the probe) at a speed of 0.5 mm/s until the maximum force of 45N was achieved. The force-distance profiles associated with the penetration of the probe into the matrices were generated at a data acquisition rate of 200 points per second.

Tablet Resistance/Force of Penetration (N) (Mean Force to the First Peak):

Tablet resistance/Force of penetration (N)

| Time (hr) | A | B | C | D |
|---|---|---|---|---|
| 0 | 21.162 | 21.189 | 21.085 | 20.715 |
| 4 | 2.855 | 13.119 | 6.36 | 14.356 |
| 8 | 1.324 | 12.021 | 3.418 | 12.446 |
| 24 | 0.805 | 8.554 | 0.733 | 3.566 |

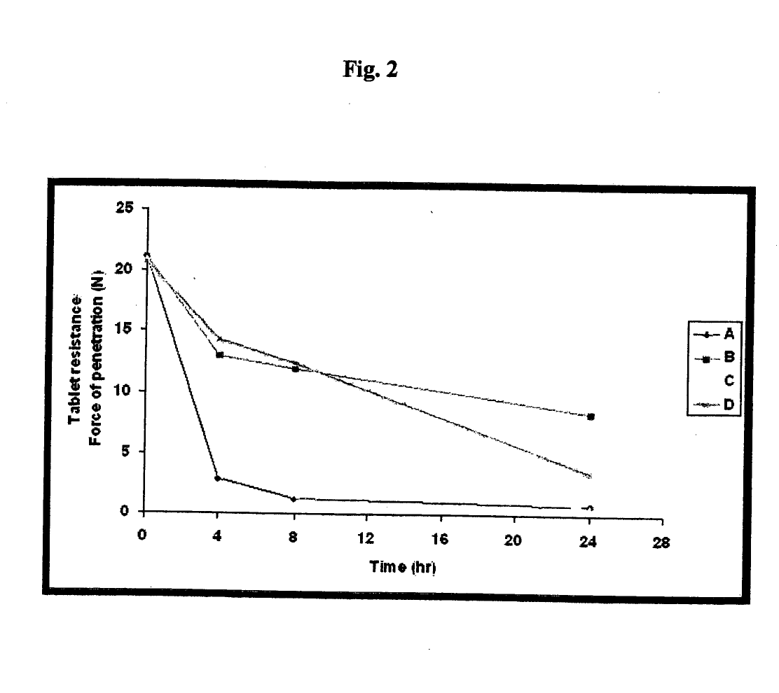

A plot of the above data is shown as FIG. 2.

c. Conclusion
   Results indicate that increasing levels of PVAP (samples B and D) form a gel layer at a slower rate than the samples which contain lactose as the predominant filler (samples A and C). This is evidenced by the higher force of penetration values for samples B and D compared to A and C. The presence of the lactose allows rapid hydration of the HPMC and formation of a gel layer through which the probe can penetrate with less resistance. Results at the 24 hour interval indicate that higher levels of PVAP in combination with HPMC provide a matrix tablet and hydrated gel layer with significant mechanical strength remaining after this time interval. This indicates that incorporation of PVAP into the matrix composition is modifying the behavior of the matrix from a diffusion/erosion based mechanism to predominantly erosion.

### Example 4

### Mass Loss Studies and Liquid Uptake Investigations

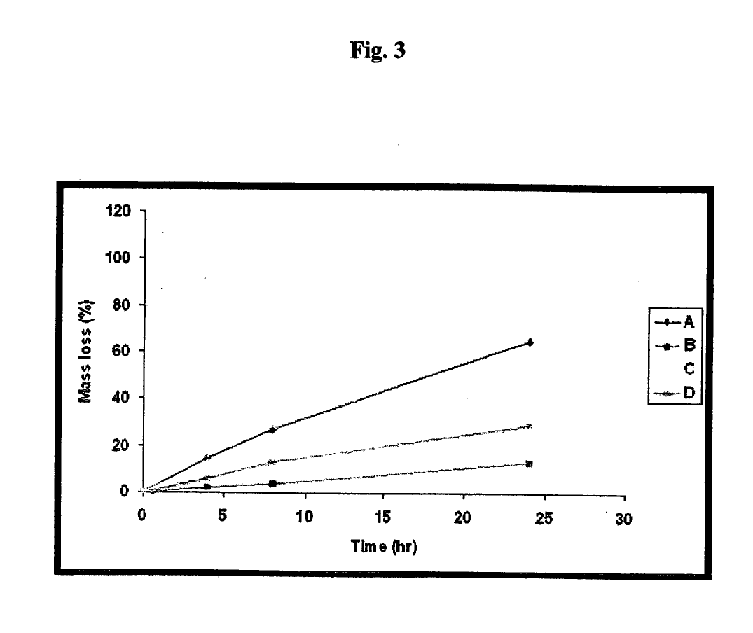

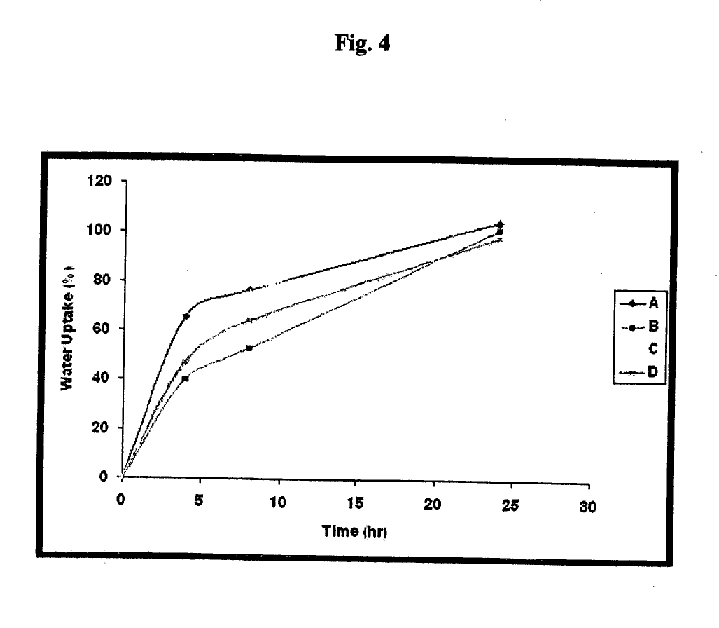

a. Composition
   PMC/PVAP compacts (5 g) with the compositions as in Example 2 were prepared using the Carver Press at the compaction force of 2500 pounds and the hold time of 15 s.
b. Method
   Same as the process of Example 2, the tablets were placed in a beaker containing deionized water. They were removed from the beaker at predetermined time points (4, 8, 24 hours) and lightly patted with a tissue paper to remove excess water. Mass loss was calculated by drying the wet compacts to constant weight, and comparing to the original weight of the dry tablet. The result is shown in FIG. 3. Liquid uptake was calculated by comparing the weight of water up taken to the tablet with the weight of dry tablets. The result is shown in FIG. 4.
c. Conclusion
   Increasing levels of PVAP in combination with HPMC has shown a reduction in the mass loss and water intake. Tablet mass loss, and liquid uptake as shown in FIG. 3, and FIG. 4 demonstrates that as the PVAP level increases, the rate of mass loss is reduced and the ingress of water is impeded. Since all formulations contain a similar level of HPMC for gel formation, the reduction of mass loss and the impeding of water ingress are associated with the synergistic interaction of HPMC and PVAP in the presence of acidic or basic pH media.

### Example 5

### Viscosity Investigation—0.1N HCl or pH 6.8 Phosphate Buffer

a. Dispersion Characterization
   PVAP, HPMC, or Verapamil HCl was dispersed in 0.1N HCl or phosphate buffer, pH 6.8. Viscosity was characterized neat and in binary or tertiary mixtures.
b. Dry Blending Mixtures Characterization
   i. 2 parts HPMC was dry blended with 30 parts PVAP and dispersed in in 0.1N HCl or pH 6.8 phosphate buffer to a final solid content of 19%.
   ii. 2 parts HPMC, 30 parts PVAP, and 48 parts Verapamil HCl were dry blended and dispersed in 0.1N HCl or pH 6.8 phosphate buffers to a final solids content of 36%.
   A Brookfield viscometer, DV-II+, equipped with RV spindles 1 and 3 were utilized for determination of viscosity.

c. Results (as summarized in following table):

| | Viscosity | Viscosity (cP) |
|---|---|---|
| | (cP) | Phosphate |
| Material | 0.1N HCl | Buffer, pH 6.8 |
| Verapamil HCl - 48% solution | 50.8 | 12.4 |
| PVAP - 30% dispersion | 58.8 | 12.1 |
| HPMC - 2% solution | 100.4 | 100.4 |
| 50 parts HPMC - 2% solution/50 parts | 60 | 50 |
| PVAP 30% dispersion (Total 16% | | |
| dispersion) | | |
| Powder blend 30 parts PVAP + 2 parts | 518.0 | 500.0 |
| HPMC (Total - 19% dispersion) | | |
| Powder blend 48 parts Verapamil HCl + 30 | 520.0 | 510.0 |
| parts PVAP + 2 parts HPMC | | |
| (Total - 36% dispersion) | | |

d. Conclusion
   The results from Example 5 indicate that a synergistic increase in dispersion viscosity is found only when PVAP and HPMC are pre-blended as a powder prior to dispersion. When the two polymers were dispersed separately and mixed, a synergistic increase in dispersion viscosity is not observed. The synergistic increase in dispersion viscosity by combining HPMC and PVAP is independent of the pH media with which they are prepared. The end result is that drug released with these combinations can be retarded in acidic, neutral, and alkaline conditions, based on the observed pH independent synergistic increase in viscosity.

### Example 6

### Dissolution Studies—Verapamil HCL 240 mg ER Formulations

a. Composition:

Ingredient Percentages

| | 1 | 2 | 3 | 4 |
|---|---|---|---|---|
| Verapamil HCl | 48 | 48 | 48 | 48 |
| Methocel K100LV | 20 | 20 | 20 | 20 |
| PVAP | 0 | 31 | 7.75 | 23.25 |
| Spray Dried Lactose | 31 | 0 | 23.25 | 7.75 |
| Magnesium Sterate | 0.5 | 0.5 | 0.5 | 0.5 |
| Colloidal Silicon Dioxide | 0.5 | 0.5 | 0.5 | 0.5 |

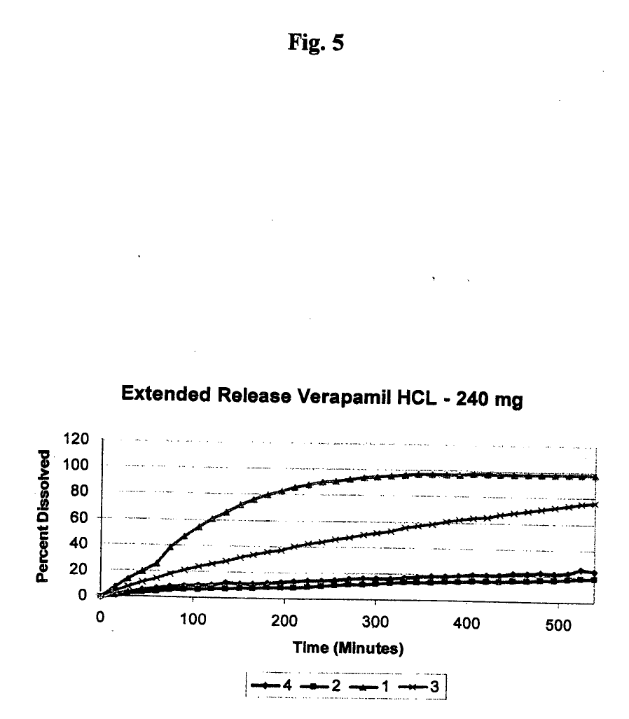

b. Method:
   Verapamil HCl (Fermion), spray dried lactose (Foremost) and/or PVAP (Colorcon) were blended in a Hobart mixer for 5 minutes and then wet-granulated with a 2% w/v Hypromellose solution (150 g, Methocel® E5, Dow Chemical Co). The wet mass was tray dried at 40° C. for 10 hours, passed through an oscillating granulator (12-mesh), and hand screened through a 16-mesh screen. The granules were then mixed with Methocel K100LV for 10 minutes in a twin shell blender. Finally, the magnesium stearate was added, and blended for an additional 3 minutes.
   500 mg tablets were manufactured using an instrumented 10 station rotary tablet press (Riva-Piccola, Argentina), fitted with 11 mm standard concave tooling, at a turret speed of 30 rpm.
   Drug release was measured (n=6) according to the USP 28 method 1 (50 rpm) using an automated dissolution bath (Varian). All methods utilized apparatus 2 (paddles), and 900 mL of simulated gastric and intestinal fluid without enzymes at 37±0.5° C. as the dissolution media. Wire helices were utilized to prevent floating of the dosage form. Drug release was measured via UV spectrophotometry at 278 nm, samples were withdrawn in the gastric phase at 60 minutes, and in the intestinal media at 120, 210, 300 and 480 minutes. The results are shown in FIG. 5.
c. Study Results:
   As shown is the FIG. 5, increasing the level of PVAP in the formulation resulted in a decrease in the release of the drug from the matrix. The interaction observed in the viscosity investigation is again shown in this example. PVAP is soluble in the intestinal media and one would therefore anticipate that if the interaction was not present, the release rate of the drug should increase from the matrix due to dissolution of the PVAP creating pathways for the drug to diffuse. This surprisingly was not the case.
d. Conclusion:
   A synergistic relationship between HPMC and PVAP is observed in acidic, alkaline, or neutral media. A similar observation is made when 240 mg Verapamil HCl ER matrices were prepared with varying levels of PVAP in the formulation. Increasing levels of PVAP resulted in a decreased release rate for the drug (especially in the pH regions corresponding to the GI tract where it was thought that PVAP would not have an effect on controlled release).

In view of the above experiments, we found that increasing levels of PVAP in combination with HPMC have shown a reduction in the drug release of Verapamil hydrochloride. Since a lack of chemical interaction has been shown between PVAP and the drug, the regulation by interaction is ruled out. Texture analysis, tablet mass loss and liquid uptake have shown that as the PVAP level increases, mass loss is reduced and the ingress of water is impeded. This corresponds to reduced conversion of the glassy core into a rubbery gel. This presents itself as a thinner gel around the matrix. This in turn alters the mechanism of release from predominantly diffusion when lactose is present, to predominantly erosion when PVAP is present. As a result, decreased mass loss and decreased drug release are observed for PVAP-containing hypomellose-based formulations. Since all formulations contain a similar level of HPMC for gel formation, the impeding of water ingress is associated with the synergistic interaction of HPMC and PVAP in the presence of water, gastric or intestinal media.

What is claimed is:

1. A controlled release formulation for use in oral dosage forms, comprising a mixture containing hypromellose and polyvinyl acetate phthalate, said polyvinyl acetate phthalate being present in amount which is effective to provide controlled release of a pharmaceutically active ingredient in vitro when said mixture is compressed into a swellable, hydrophilic matrix.

2. The controlled release formulation of claim 1, further comprising an anionic polymer.

3. The controlled release formulation of claim 2, wherein said anionic polymer is selected from the group consisting of sodium carboxymethylcellulose, sodium alginate, xanthan gum, Carbopol (cross-linked acrylic acid polymers), cellulose acetate phthalate, hydroxypropyl-methylcellulose phthalate, methacrylic acid copolymer, hydroxyppropylmethyl acetate succinate, and mixtures thereof.

4. The controlled release formulation of claim 1, further comprising a pharmaceutically active ingredient or a nutritional supplement.

5. The controlled release formulation of claim 1, further comprising an auxiliary hydrophilic cellulosic polymer.

6. The controlled release formulation of claim 5, wherein said auxiliary hydrophilic cellulosic polymer is selected from the group consisting of hydroxypropylcellulose, hydroxyethylcellulose, polyvinyl acetate and mixtures thereof.

7. The controlled release formulation of claim 1, wherein the amount of hypromellose is from about 8 to about 60% by wt.

8. The controlled release formulation of claim 7, wherein the amount of hypromellose is from about 15 to about 45% by wt.

9. The controlled release formulation of claim 8, wherein the amount of hypromellose is from about 25 to about 35% by wt.

10. The controlled release formulation of claim 1, wherein the amount of said polyvinyl acetate phthalate is from about 4 to about 60% by wt. of the mixture.

11. The controlled release formulation of claim 10, wherein the amount of said polyvinyl acetate phthalate is from about 8 to about 45% by wt. of the mixture.

12. The controlled release formulation of claim 11, wherein the amount of said polyvinyl acetate phthalate is from about 15 to about 35% by wt. of the mixture.

13. The controlled release formulation of claim 1, wherein the polyvinyl acetate phthalate is co-processed with titanium dioxide.

14. The controlled release formulation of claim 5, where the amount of auxiliary hydrophilic cellulosic polymer ranges from >0 up to about 100 percent by weight of anionic polymer.

15. The controlled release formulation of claim 1, further comprising a member of the group consisting of lubricants, flow aids, diluents, binding agents, disintegrants, binders, solubility enhancers, pH modulating agents and mixtures thereof.

16. The controlled release formulation of claim 4, wherein the pharmaceutically active ingredient or a nutritional supplement is from about 0.001 to about 60% by weight of the mixture.

17. The controlled release formulation of claim 16, wherein the pharmaceutically active ingredient or a nutritional supplement is from about 5.0 to about 40% by weight of the mixture.

18. The controlled release formulation of claim 17, wherein the pharmaceutically active ingredient or a nutritional supplement is from about 10 to about 30% by weight of the mixture.

19. The controlled release formulation of claim 1, wherein the hypromellose and polyvinyl acetate phthalate are wet granulated with a pharmaceutically active ingredient.

20. The controlled release formulation of claim 15, wherein the lubricant is selected from the group consisting of stearic acid, calcium, magnesium stearate, poloxamer, polyethylene glycol, hydrogenated vegetable oil, and mixtures thereof.

21. The controlled release formulation of claim 15, wherein the flow aid is selected from the group consisting of colloidal silicon dioxide, talc, magnesium stearate, polyethylene glycol, magnesium stearate and mixtures thereof.

22. The controlled release formulation of claim 15, wherein the diluent is selected from the group consisting of microcrystalline cellulose, lactose, dicalcium phosphate, pregelatinized starch, native starch, mannitol, sucrose, talc and mixtures thereof.

23. The controlled release formulation of claim 15, wherein the disintegration aid is selected from the group consisting of crospovidone, croscarmellose sodium, sodium starch glycolate, hydroxypropylcellulose (low-substituted), starch, calcium carbonate, carboxymethylcellulose calcium, and mixtures thereof.

24. The controlled release formulation of claim 15, wherein the solubility enhancer is selected from the group consisting of lecithin, poloxamer, polyoxyethylene-fatty acid esters, sorbitan esters, and mixtures thereof.

25. The controlled release formulation of claim 15, wherein the pH modulating agent is selected from the group consisting of citric acid, fumaric acid, tartaric acid, sodium citrate, sodium tartrate, sodium bicarbonate and mixtures thereof.

26. The controlled release formulation of claim 15, wherein the member of said group is present in an amount of from about 0.001 to about 50% by weight of the mixture.

27. The controlled release formulation of claim 4, wherein said hypromellose and said polyvinyl acetate phthalate are dry blended prior to being mixed with said pharmaceutical active ingredients or said nutritional supplementary.

28. The controlled release formulation of claim 4, wherein said dry blend of said hypromellose and said polyvinyl acetate phthalate are dispersed in an aqueous solution prior to being combined with said pharmaceutical active ingredients or said nutritional supplementary.

29. An oral solid dosage form comprising the controlled release formulation of claim 1.

30. A method of preparing an oral solid dosage form, comprising providing the controlled release formulation of claim 4 and compressing the formulation into an oral solid dosage form.
